(19) United States
(12) Patent Application Publication
(10) Pub. No.: US 2019/0192409 A1
(43) Pub. Date: Jun. 27, 2019

(54) OILY OXIDIZING AGENT COMPOSITIONS IN PACKAGES MADE OF BARRIER FILM

(71) Applicant: Henkel AG & Co. KGaA, Duesseldorf (DE)

(72) Inventors: Marc Nowottny, Moenchengladbach (DE); Burkhard Mueller, Duesseldorf (DE)

(73) Assignee: Henkel AG & Co. KGaA, Duesseldorf (DE)

(21) Appl. No.: 16/331,894

(22) PCT Filed: Jun. 30, 2017
(86) PCT No.: PCT/EP2017/066251

§ 371 Date: Mar. 8, 2019

(30) Foreign Application Priority Data
Sep. 9, 2016 (DE) 10 2016 217 180.1

(51) Int. Cl. A61K 8/58; A61Q 5/10; A61Q 5/08; A61K 8/22; A61K 8/46; A61K 8/92; A61K 8/86; A61K 8/368; A61K 8/34; B65D 65/40; B65D 75/28

(52) U.S. Cl. CPC A61K 8/58; A61Q 5/10; A61Q 5/08; A61K 8/22; A61K 8/463; A61K 8/922; A61K 2800/4322; A61K 8/368; A61K 8/342; B65D 65/40; B65D 75/28; A61K 2800/87; A61K 2800/4324; A61K 8/86

(57) ABSTRACT

The present disclosure relates to a cosmetic product for changing the natural color of keratinic fibers, in particular human hair, which contains at least one package (VP) and a cosmetic composition (KM) located in this package (VP). The package is made of a multi-layer film (F) which contains at least two polymer layers (P1) and (P2) and at least one barrier layer (BS). The cosmetic composition comprises at least one oxidizing agent, at least one C₈-C₃₀ alcohol, at least one anionic surfactant, at least one nonionic surfactant and at least one liquid cosmetic oil. The use of the package (VP) in combination with the cosmetic composition (KM) surprisingly does not lead to a swelling of the package or excessive water loss of the agent (KM) during storage.

### CROSS-REFERENCE TO RELATED APPLICATION

This application is a U.S. National-Stage entry under 35 U.S.C. § 371 based on International Application No. PCT/EP2017/066251, filed Jun. 30, 2017, which was published under PCT Article 21(2) and which claims priority to German Application No. 10 2016 217 180.1, filed Sep. 9, 2016, which are all hereby incorporated in their entirety by reference.

### TECHNICAL FIELD

The present disclosure is in the field of cosmetics and relates to a product for the oxidative color change of keratinic fibers, in particular human hair, which product comprises an oxidizing agent-containing composition packaged in a package. The oxidizing agent-containing composition contains at least one C₈-C₃₀ alcohol, at least one nonionic and one anionic surfactant and at least one cosmetic oil. The package is a package made of a special multi-layer film composite system, the wall of which comprises at least two polymeric layers and a barrier layer. The barrier layer here has a passage barrier effect for gases and water vapor.

### BACKGROUND

The change in the color of keratinic fibers, in particular hair, represents an important area of modern cosmetics. As a result, the appearance of the hair can be adapted to both current fashion trends and the individual wishes of the individual. The person skilled in the art knows different possibilities for changing the color of hair.

Hair color can be changed temporarily through the use of direct acting dyes. In this case, already fully formed dyes diffuse from the colorant into the hair fiber. The dyeing with direct acting dyes is associated with little hair damage, but a disadvantage is the low durability and fast washability of the colorings obtained with direct acting dyes.

Thus, when the consumer wishes to have a long-lasting color result or a shade lighter than his original hair color, oxidative color-change agents are usually used. So-called oxidation colorants are used for permanent, intensive dyeings with corresponding fastness properties. Such colorants usually contain oxidation dye precursors, so-called developer components and coupler components which, under the influence of oxidizing agents, usually hydrogen peroxide, form the actual dyes with one another. Oxidation colorants are exemplified by excellent, long-lasting coloring results.

The pure lightening or whitening of hair is often done by using oxidizing agents without the addition of oxidation dye precursors. For a medium whitening effect, the use of hydrogen peroxide alone is sufficient as the oxidizing agent, for the achievement of a stronger whitening effect, a mixture of hydrogen peroxide and peroxide sulfate salts is usually used.

Oxidative color-changing agents are usually marketed in the form of two-component agents, in which two different preparations are assembled separately in two separate packages and are mixed together just shortly before use.

The first preparation is a formulation (usually acidified for reasons of stability) which contains, as oxidizing agent, for example, hydrogen peroxide in concentrations of 1.5 to 12% by weight. The oxidizing agent formulation is usually present in the form of an emulsion or dispersion and is usually provided in a plastic bottle having a resealable outlet opening (developer bottle).

This oxidizing agent formulation is mixed with a second formulation prior to use. This second preparation is an alkaline formulation which is often present in the form of a cream or gel and which also contains at least one oxidation dye precursor when a color change is desired at the same time as the lightening. This second preparation can be provided, for example, in the form of a tube or in the form of a plastic or glass container.

In the conventional application described above, the second preparation, which contains the alkalizing agent and/or the oxidation dye precursors, is transferred from the tube or container into the developer bottle and then mixed by shaking with the hydrogen peroxide preparation already located in the developer bottle. In this way, the application mixture is prepared in the developer bottle. The application on the hair then takes place via a small spout or outlet opening at the head of the developer bottle. The spout or outlet opening is opened after shaking and the application mixture can be removed by pressing the flexible developer bottle.

The use of the developer bottle requires a certain amount of routine from the user, so that some users prefer to make the application mixture in a mixing bowl and apply by employing a brush.

When preparing the application mixture in a bowl, both components, the first preparation containing the oxidizing agent and the second preparation with alkalizing agent and/or oxidation dye precursors, are completely transferred to a bowl or a similar vessel and stirred there, for example, with the aid of a brush. The application mixture is then removed via the brush from the mixing bowl. In this form of application, the use of a voluminous and expensive developer bottle is not necessary, and it is sought after as an inexpensive and material-saving packaging form for the oxidizing agent preparation.

In this context, packages in the form of a bag or a pouch can be used as an inexpensive packaging form with low material consumption, which bag or pouch is usually made of plastic films or metal foils.

Such a package can be produced, for example, by bonding or hot-pressing two plastic films lying one on top of the other, wherein the bonding takes place on all edges of the films. The interior of the package (that is, the plastic bag) produced by bonding can then be filled with the desired cosmetic preparation. The package can be opened by tearing or cutting the plastic bag.

However, the filling of oxidizing agent preparations in such packages is associated with problems whose cause is due to the reactivity of the oxidizing agent. Oxidizing agents are highly reactive substances which, depending on the storage conditions and possibly on the presence of decomposing active impurities, decompose in small amounts to form oxygen (that is, gas).

The developer bottles known from the state of the art are usually filled with the oxidizing agent composition at most only one half, usually only one third, of their internal volume. As a rule, developer bottles are made of polyethylene. Since polyethylene is permeable with respect to both water vapor and gases, no or very little overpressure arises in the developer bottle. In addition, developer bottles are usually provided with sturdy, thick walls and a sturdy screw-on closure, so that the diffusion of water vapor or gases through the thickness of the walls is reduced and a slight pressure increase taking place within the bottle has no negative effects.

In contrast, bag-shaped packages, however, are usually completely filled with the liquid preparation, and there is virtually no supernatant airspace in the filled bag. In addition, such packages should be flexible, and when opening (for example, tearing or slicing), no uncontrolled discharge of the preparation should occur. For this reason, in the packaging of liquid preparations, the emergence of overpressure in the package should be avoided as far as possible.

If an oxidizing agent composition is present in such a package, the gas (oxygen) produced during storage can cause the package to swell. Since the edges of the package are usually only glued, a strong swelling at worst leads to bursting of the package. For these reasons, when storing oxidizing agent-containing compositions, the choice of the film material from which the package is made is of great importance.

Packaging that is made of pure plastic, such as polyethylene or polypropylene, is permeable with respect to both water vapor and gases. No swelling of the package therefore occurs when storing an oxidizing agent-containing preparation in a package made of polyethylene or polypropylene. Due to the high permeability of the relatively thin film of the package with respect to water vapor, however, the water content of the preparation is reduced. If the preparation is stored in the package for a few weeks to months, the loss of water exceeds the maximum value permitted for sufficient storage stability.

Completely airtight packages are made, for example, from plastic films which have a lamination with a metal layer, for example, with an aluminum layer. These packages are impermeable with respect to water vapor and gases. If these packages are filled with an oxidizing agent-containing preparation, the gas produced during the decomposition of the oxidizing agent cannot escape, the package swells as described above and can burst.

### BRIEF SUMMARY

Cosmetic products for changing the natural color of keratinic fibers are provided herein. In an embodiment, a cosmetic product for changing the natural color of keratinic fibers includes:

(i) a package, which includes a multi-layer film, with the mulit-layer film including a first polymer layer, a second polymer layer and a barrier layer, and

(ii) a cosmetic composition packed in the package and including:
a) an oxidizing compound,
b) a C₈-C₃₀ alcohol,
C) an anionic surfactant,
d) a nonionic surfactant and
e) a liquid cosmetic oil.

In another embodiment, a cosmetic product for changing the natural colour of keratinous fibres includes:

(i) a package, including a multi-layer film, which includes:
a first polymer layer including polypropylene and having a layer thickness of from about 60.0 μm to about 90.0 μm,
a second polymer layer including polyethylene terephthalate and having a layer thickness of from about 10.0 μm to about 20.0 μm, and
a barrier layer including:
   a layer including silicon dioxide; and
   a layer including an inorganic-organic hybrid polymer;
wherein the barrier layer has a layer thickness of from about 10.0 nm to 150 nm and is disposed between the first polymer layer and the second polymer layer, and

(ii) a cosmetic composition, which is packaged in the package and includes:
a) an oxidizing compound including hydrogen peroxide and present in a total quantity of from about 1.5 to about 15% by weight, in relation to the total weight of the cosmetic composition,
b) a mixture of linear C₁₄-C₁₈ alcohols and is present in an amount of from about 1.5 to about 5.0% by weight, with respect to the total weight of the cosmetic composition;
c) sodium cetearyl sulfate present in a total quantity of from about 0.20 to about 1.5% by weight, based on the total weight of the cosmetic composition;
d) PEG-40 hydrogenated castor oil present in a total quantity of from about 0.30 to about 1.5% by weight, based on the total weight of the cosmetic composition;
e) a liquid cosmetic oil present in a total quantity of from about 5.0 to about 20% by weight, based on the total weight of the cosmetic composition; and
f) water present in a total quantity of from about 60 to about 97% by weight, in relation to the total weight of the cosmetic composition.

In another embodiment, a cosmetic product for changing the natural colour of keratinous fibres includes:

(i) a package, including a multi-layer film, which includes:
a first polymer layer including polypropylene and having a layer thickness of from about 60.0 μm to about 90.0 μm,
a second polymer layer including polyethylene terephthalate and having a layer thickness of from about 10.0 μm to about 20.0 μm, and
a barrier layer including:
   a layer including silicon dioxide; and
   a layer including an inorganic-organic hybrid polymer;
wherein the barrier layer has a layer thickness of from about 10.0 nm to 150 nm and is disposed between the first polymer layer and the second polymer layer, and

(ii) a cosmetic composition, which is packaged in the package and consists of:
a) an oxidizing compound,
b) a mixture of linear C₁₄-C₁₈ alcohols;
c) an inorganic alkalizing agent;
d) sodium cetearyl sulfate;
e) PEG-40 hydrogenated castor oil
f) water present in a total quantity of from about 60 to about 97% by weight, in relation to the total weight of the cosmetic composition; and
g) a mixture of acids including:
   at least one of dipicolinic acid, disodium pyrophosphate, ethylenediamine tetraacetic acid, or salts thereof;
   1-hydroxyethane-1,1-diphosphonic acid; and
   benzoic acid or a salt thereof.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present application was to package the oxidizing agent composition in an inexpensive, material-saving, space-saving, safe and in particular storage-stable manner.

Surprisingly, it has now been found that oxidizing agent-containing compositions can be packaged in a storage-stable manner when, on the one hand, special packages are used, which packages includes special film composite systems and additionally have a barrier layer. On the other hand, the oxidizing agent preparation can be further stabilized by the combination of at least one C₈-C₃₀ alcohol, at least one anionic and nonionic surfactant and at least one cosmetic oil.

The present disclosure is a cosmetic product for changing the natural color of keratinic fibers, in particular human hair, comprising
(i) at least one package (VP), comprising at least one multi-layer film (F), which contains at least one first polymer layer (P1), at least one second polymer layer (P2) and at least one barrier layer (BS), and
(ii) at least one cosmetic composition (KM) packed in the package (VP) and contains:
   a) at least one oxidizing compound,
   b) at least one C₈-C₃₀ alcohol,
   c) at least one anionic surfactant,
   d) at least one nonionic surfactant and
   e) at least one liquid cosmetic oil.

Keratinic fibers, keratin-containing fibers or keratin fibers are understood to mean furs, wool, feathers and, in particular, human hair. Although the agents as contemplated herein are primarily suitable for lightening and dyeing keratin fibers, in principle, there is nothing to prevent their use in other fields as well.

The product as contemplated herein is a product for the oxidative color change of keratinic fibers, that is, a product which is applied to the human head to achieve oxidative dyeing, lightening, whitening, bleaching or shading of the hair. In this context, “shading” is understood to mean a dyeing in which the color result is lighter than the original hair color.

Furthermore, the term “package” as contemplated herein is understood to mean a package which is preferably present in the form of a sachet. A sachet is a small package in bag or pouch form, which is often used in the packaging of cosmetics. The capacity of the package, in particular of the sachet, can be, for example, from about 5 to about 1000 ml, preferably from about 10 to about 200 ml and particularly preferably from about 20 to about 50 ml.

In addition, a multi-layer film (F) in the context of the present disclosure is understood to mean a thin, laminar and windable web of the at least one polymer layer (P1) and the at least one polymer layer (P2). This multi-layer film (F) forms the wall of the package (VP). The polymer layers (P1) and (P2) preferably comprise polymers capable of forming films. Furthermore, the polymer layers (P1) and (P2) are preferably polymer layers different from each other. The package additionally contains a barrier layer (BS) which prevents or reduces the passage of water vapor and other gases, such as oxygen, thus preventing or reducing the diffusion of these gases through the wall of the package.

The term “anionic surfactant” as contemplated herein is understood to mean amphiphilic (bifunctional) compounds, which includes or, alternatively consist of, at least one hydrophobic and at least one hydrophilic molecule part The hydrophobic radical is preferably a hydrocarbon chain having from about 8 to about 28 carbon atoms, which can be saturated or unsaturated, linear or branched. Particularly preferably, this C₈-C₂₈ alkyl chain is linear. In addition, these surfactants contain at least one anionic group, in particular a carboxylate and/or sulfonate group.

Furthermore, the term “nonionic surfactant” as contemplated herein is understood to mean amphiphilic (bifunctional) compounds which have at least one hydrophobic and at least one hydrophilic part. The hydrophobic radical is preferably a hydrocarbon chain having 8 to 28 carbon atoms, which can be saturated or unsaturated, linear or branched. Particularly preferably, this C₈-C₂₈ alkyl chain is linear. In contrast to anionic, cationic, zwitterionic and amphiphilic surfactants, nonionic surfactants contain neither cationic nor anionic groups. In addition, these surfactants also have no cationizable and anionizable groups which can form cationic or anionic groupings, depending on the pH value.

Finally, the term “liquid cosmetic oils” in the context of the present disclosure is understood to mean oils suitable for cosmetic use which are insoluble in water at 20° C., that is, dissolve in water at 20° C. preferably less than 1% by weight of the oil, based on the total amount of the water-oil mixture. However, the cosmetic oil used as contemplated herein is neither fragrance nor essential oils. In the context of the present disclosure, paraffin oils are preferably used as cosmetic oils.

The cosmetic product as contemplated herein comprises as the first component a package (VP) which comprises at least one multi-layer film (F). This film contains at least one first polymer layer (P1), at least one second polymer layer (P2) and at least one barrier layer (BS). This multi-layer film represents the wall or the outer shell of the package. As described above, such a package is usually made by gluing, pressing or sealing two superimposed pieces of film (wherein the package (VP) is filled simultaneously with the cosmetic composition (KM)), that is, such a package is closed at all edges. This package can be opened, for example, by tearing or cutting open.

The thickness of the multi-layer film (F) should in this case be designed so that a sufficient mechanical stability is present, but at the same time, the film (F), and thus the package produced from the film (VP), is so flexible that a complete removal the cosmetic composition (KM) from the opened package (VP) by pressing or pressing is enabled. These requirements are met in particular when the film (F) has a certain total thickness. Preferred embodiments of the present disclosure are therefore exemplified in that the at least one multi-layer film has a total thickness of from about 21 μm to about 2.0 mm, preferably from about 30 μm to about 1.0 mm, more preferably from about 50 μm to about 500 μm, in particular from about 60 μm to about 200 μm. For the purposes of the present disclosure, the total thickness of the film (F) is understood to mean the sum of the thicknesses of all the individual layers of the film (F).

The arrangement of the layers (P1), (P2) and (BS) within the multi-layer film (F) can be different. Furthermore, it is also possible for the film (F) to comprise further layers in addition to the previously mentioned layers. In addition, it is advantageous as contemplated herein when all of the previously mentioned layers are each oriented parallel to the surfaces of the film (F), that is, all layers have the same orientation.

It is particularly preferred as contemplated herein when the barrier layer (BS) is arranged on the side in contact with the cosmetic composition (KM). The first polymer layer (P1) thus adjoins firstly the barrier layer (BS) and secondly the second polymer layer (P2), which is located on the outside of the package. The polymer layer (P1) here is different from the polymer layer (P2). Here, the barrier layer (BS) serves as a carrier layer, to which then the first polymer layer (P1) is applied. The second polymer layer (P2) is then applied to this polymer layer (P1). The three layers (BS), (P1) and (P2) together form a film (F) whose total thickness is preferably from about 30 μm to about 1.0 mm.

However, in the context of the present disclosure, an arrangement in which the barrier layer (BS) lies between the first polymer layer (P1) and the second polymer layer (P2) is particularly preferred. In this case, the multi-layer film (F) includes or, alternatively, consists of, three layers, wherein the layer (P1) lies in the innermost contact with the cosmetic composition (KM). The layer (P1) is in contact with the barrier layer (BS), and the barrier layer (BS) in turn makes contact with the layer (P2). In this layer, the layers (P1) and (P2) do not adjoin one another but rather are separated by the barrier layer (BS). In this arrangement, the layers (P1) and (P2) can in principle be made of the same polymeric material, but it is preferred when the two layers (P1) and (P2) are made of different polymeric materials. The three layers (BS), (P1) and (P2) together form a film (F) whose total thickness is preferably from about 30 μm to about 1.0 mm. The particular advantage of this arrangement is that the, often very thin, barrier layer (BS) is located neither on the inner nor on the outer surface of the multi-layer film (F), but rather is protected in the direction of the inside through the polymeric layer (P1) and in the direction of the outside by the polymeric layer (P2). In this way, in this arrangement, a mechanical abrasion or mechanical destruction of the barrier layer (BS) is best avoided. It is therefore advantageous in the context of the present disclosure for the at least one multi-layer film (F) to contain the at least one barrier layer (BS) between the at least one first polymer layer (P1) and the at least one second polymer layer (P2). The use of such packages has been found to be particularly advantageous in terms of increased storage stability since this arrangement exhibits neither swelling nor delamination with prolonged contact time with an oxidizing agent-containing composition.

Also particularly preferred as contemplated herein is a film (F) in which the first polymer layer (P1) is arranged on the side in contact with the cosmetic composition (KM). The second polymer layer (P2) adjoins the polymer layer (P1) and is different from this. The barrier layer (BS) is located outside. For films (F) with this layering, for example, the layer (P1) can function as a polymeric carrier layer onto which the second polymeric layer (P2) is then applied. Subsequently, the side adjacent to (P2) (that is, the outside) is provided with the barrier layer. It is therefore advantageous in the context of the present disclosure for the at least one multi-layer film (F) to contain the at least one barrier layer (BS) on the outside of the package (VP). As contemplated herein, the outside of the package (VP) is understood to mean that side of the package which does not come into contact with the cosmetic composition (KM) but rather with the environment. The three layers (P1), (P2) and (BS) in this case form a film (F) whose total thickness is preferably from about 30 μm to about 1.0 mm. The use of such packages has been found to be particularly advantageous in terms of increased storage stability since this arrangement exhibits neither swelling nor delamination with prolonged contact time with an oxidizing agent-containing composition.

If the multi-layer film (F) contains the above-described three layers (P1), (P2) and (BS), suitable arrangements as contemplated herein of the layers are described below (considered from interior (in contact with the cosmetic composition (KM)) to the outside):

a) *Interior*-layer (P1)-layer (P2)-barrier layer (BS)-*outside*,
b) *Interior*-layer (P1)-barrier layer (BS)-layer (P2)-*outside*,
c) *Interior*-layer (P2)-layer (P1)-barrier layer (BS)-*outside*,
d) *Interior*-layer (P2)-barrier layer (BS)-layer (P1)-*outside*,
e) *Interior*-barrier layer (BS)-layer (P1)-layer (P2)-*outside*,
f) *Interior*-barrier layer (BS)-layer (P2)-layer (P1)-*outside*,

The first polymeric material of the first layer (P1) is as contemplated herein an organic polymeric material. This material can be a polymer type layer or a polymer blend layer. This first layer (P1) can, for example, function as a polymeric carrier material, that is, in the production of the film, a layer or a film of the polymeric material (P1) can be initially furnished and then sprayed, laminated or coated with the further layers as contemplated herein. Preferred embodiments of the present disclosure are exemplified in that the at least one first polymer layer (P1) is formed from polypropylene, polyethylene, polyester, polyamide or polyvinyl alcohol, in particular from polypropylene. The term “is formed” is understood as contemplated herein to mean that the polymer layer contains at least about 70% by weight, preferably at least about 80% by weight, preferably at least about 90% by weight, in particular at least about 99% by weight, each based on the total weight the polymer layer (P1), of the previously mentioned compounds.

A particularly preferred product as contemplated herein is therefore exemplified in that the multi-layer film (F) comprises at least one first polymer layer (P1) which is formed from polypropylene. Polypropylene is alternatively referred to as poly(l-methylethylene), and is a thermoplastic polymer which belongs to the group of polyolefins. Polypropylene is made by polymerizing propylene (propene) using various catalysts. For example, polypropylene can be produced by stereospecific polymerization of propylene in the gas phase or in suspension according to Giulio Natta. Polypropylenes as contemplated herein can be isotactic and thus highly crystalline, but also syndiotactic or amorphous. The regulation of the average relative molar mass can be effected, for example, by setting a specific hydrogen partial pressure during the polymerization of the propene. For example, polypropylene can have average relative molecular weights of from about 150,000 to about 1,500,000 g/mol. Polypropylene can be processed, for example, by extrusion and stretch blow molding, or by pressing, calendering, thermoforming and cold forming.

The first polymer layer (P1) preferably has a specific layer thickness. It is therefore preferred in the context of the present disclosure when the at least one first polymer layer (P1) has a layer thickness of from about 20.0 μm to about 300 μm, preferably from about 40.0 μm to about 200 μm, more preferably from about 50.0 μm to about 100 μm, in particular from about 60.0 μm to about 90.0 μm.

A particularly preferred product as contemplated herein is therefore exemplified in that multi-layer film (F) comprises at least one first polymer layer (P1), which is formed from polypropylene and has a layer thickness of from about 60.0 to about 90.0 μm.

Furthermore, the multi-layer film (F) from which the package is made comprises a second polymer layer (P2) of a second polymeric material. The second polymeric material can be a polymer type layer or a polymer blend layer. In the production of the multi-layer film, for example, the second layer (P2) can be sprayed, applied or coated either before or after application of the barrier layer (BS) to the first polymer layer (P1) acting as the carrier layer. However, it is also conceivable that the second polymer layer (P2) acts as a carrier layer, to which the barrier layer (BS) and the first polymer layer (P1) are then applied.

Depending on the sequence of layering described above, the first polymeric material of the first polymer layer (P1) and the second polymeric material of the second polymer layer (P2) can either be the same (if both layers are not in contact with each other) or can be different. The polymer layer (P2) can therefore be formed from the compounds previously mentioned in connection with the polymer layer (P1). Preferably, the layers (P1) and (P2) are made of different polymeric materials (that is, different polymers or polymer blends). It is therefore preferred within the context of the present disclosure for the at least one second polymer layer (P2) to be formed from polyethylene terephthalate or polyethylene naphthalate, in particular from polyethylene terephthalate. The term “is formed” is understood as contemplated herein to mean that the polymer layer contains at least about 70% by weight, preferably at least about 80% by weight, preferably at least about 90% by weight, in particular at least about 99% by weight, each based on the total weight the polymer layer (P2), of the previously mentioned compounds. Polyethylene terephthalate (PET) is a polymer from the group of polyesters. The preparation of polyethylene terephthalate can be carried out, for example, by transesterification of dimethyl terephthalate with ethylene glycol at higher temperatures. Methanol is split off in this transesterification reaction, which is removed by distillation. The resulting bis(2-hydroxyethyl) terephthalate is converted by polycondensation to PET, wherein ethylene glycol is produced again. A further production method of polyethylene terephthalate is the direct polycondensation of ethylene glycol and terephthalic acid at high temperatures while distilling off the resulting water.

Preferably, the second polymer layer (P2) has a smaller layer thickness than the polymer layer (P1). It is therefore preferred in the context of the present disclosure when the at least one second polymer layer (P2) has a layer thickness of from about 1.00 μm to about 100 μm, preferably from about 2.50 μm to about 50.0 μm, more preferably from about 5.00 μm to about 25.0 μm, in particular from about 10.0 μm to about 20.0 μm.

A particularly preferred product as contemplated herein is therefore exemplified in that multi-layer film (F) comprises at least one second polymer layer (P2), which is formed from polyethylene terephthalate and has a layer thickness of from about 10.0 to about 20.0 μm.

The polymer layers (P1) and (P2) of the multi-layer film (F) include or, alternatively, consist of, organic polymeric materials, which usually have only an insufficient barrier effect with respect to gases and water vapor. If the oxidizing agent-containing composition (KM) is packaged in a package (VP) of a multi-layer film (F), which comprises only the two organic polymer layers (P1) and (P2), water vapor can escape unhindered, so that the water content in the composition (KM) changes unacceptably during prolonged storage. In order to minimize the uncontrolled escape of water vapor from the package (VP), the organic polymer layers (P1) and (P2) are therefore used in conjunction with a barrier layer (BS).

The barrier layer (BS) has a passage barrier effect for gases and water vapor. As contemplated herein, it is meant that the barrier layer (BS) reduces the permeation rate of water vapor and gases through the film. A film (F) as contemplated herein, which has a barrier layer (BS) in addition to the layers (P1) and (P2), thus has with respect to a comparable film (with the same total thickness), which however only has the two layers (P1) and (P2) but has no barrier layer (BS), a reduced water vapor permeability and reduced gas permeability.

By way of example, the barrier layer (BS) is a thin layer which comprises an inorganic material, wherein the inorganic material can be applied to the organic polymer layer (P1) and/or (P2) by employing vacuum coating techniques (for example, PVD “physical vapor deposition” or CVD “chemical vapor deposition”).

If the barrier layer (BS) is a layer which comprises at least one inorganic material, then, for example, aluminum, aluminum oxides, magnesium, magnesium oxides, silicon, silicon oxides, titanium, titanium oxides, tin, tin oxides, zirconium, zirconium oxide and/or or carbon can be considered. Particularly preferred in this context are oxides which can be selected from the group of aluminum oxides, magnesium oxides, silicon oxides, titanium oxides, tin oxides and/or zirconium oxides. The barrier layer (BS) of inorganic material is very particularly preferably between the two polymer layers (P1) and (P2). The production of films with barrier layers of inorganic material is described, for example, in document EP 1036813 A1, to which reference is made in full at this point.

The barrier layer (BS) can also comprise a thin layer of inorganic-organic hybrid polymers. These polymers are known in the literature under the technical term ORMOCER polymers. A typical ORMOCER polymer can be prepared, for example, by hydrolytic polycondensation of an organofunctional silane with an aluminum compound and optionally with an inorganic oxide component. Corresponding syntheses are disclosed, for example, in the document EP 0792846 B1, to which reference is made in full at this point. Inorganic-organic hybrid polymers (ORMOCER polymers) have both inorganic and organic network structures. The structure of the inorganic silicate network structure can be carried out in the sol-gel process via the controlled hydrolysis and condensation of alkoxysilanes. The silicate network can be modified in a targeted manner by additionally incorporating metal alkoxides into the sol-gel process. An organic network is additionally built by polymerization of organofunctional groups which are introduced by the organoalkoxysilanes into the material. The ORMOCER polymers produced in this way can be applied to the layers (P1) and/or (P2), for example, by employing conventional application techniques (spraying, brushing, etc.).

Preferred embodiments of the present disclosure are therefore exemplified in that the at least one barrier layer (BS) is formed from aluminum oxides, magnesium oxides, silicon oxides, titanium oxides, tin oxides, zirconium oxides, inorganic-organic hybrid polymers (ORMOCER polymers) or mixtures thereof, in particular of silicon oxides. The term “is formed” is understood as contemplated herein to mean that the polymer layer contains at least about 70% by weight, preferably at least about 80% by weight, preferably at least about 90% by weight, in particular at least about 99% by weight, each based on the total weight the barrier layer (BS), of the previously mentioned compounds. Particularly preferred are multi-layer films (F) as contemplated herein in which the barrier layer (BS) is formed from silicon oxides or inorganic-organic hybrid polymers (ORMORCER polymers).

Furthermore, it is also possible that the multi-layer film (F), which represents the wall of the package (VP), has a barrier layer (BS) which comprises both inorganic oxide components and inorganic-organic hybrid polymers (ORMOCER polymers). In addition, the barrier layer (BS) can also comprise a further organic polymeric material which itself has no barrier effect but, for example, increases the mechanical stability of the barrier layer, simplifies manufacture or causes better bonding of the layers (BS) and (P1) and/or (P2). Particularly preferred are multi-layer films (F) as contemplated herein in which the barrier layer (BS) is formed from aluminum oxides, magnesium oxides, silicon oxides, titanium oxides, tin oxides, zirconium oxides and mixtures thereof and additionally at least one inorganic-organic hybrid polymer (ORMORCER polymers).

The thicker the barrier layer (BS), the greater or stronger the passage barrier effect for gases and water vapor. The thickness of the barrier layer (BS) can therefore be chosen as a function of the desired barrier effect. The barrier layer (BS) can have, for example, a layer thickness of from about 1 to about 1000 nm (nanometers). The barrier layer (BS) preferably has a layer thickness of from about 5 to about 500 nm, more preferably of from about 10 to about 250 nm and particularly preferably of from about 10 to about 150 nm (nanometers). Preferred embodiments of the present disclosure are therefore exemplified in that the at least one barrier layer (BS) has a layer thickness of from about 1.00 nm to about 1000 nm, preferably from about 5.00 nm to about 500 nm, more preferably from about 10.0 nm to about 250 nm, in particular from about 10.0 nm to about 150 nm.

In addition to the previously described layers (P1), (P2) and (BS), the multi-layer film (F) can additionally comprise one or more further layers. These further layers can be, for example, intermediate layers and/or adhesive layers. It is therefore preferred as contemplated herein when the at least one multi-layer film (F) additionally contains at least one further layer selected from the group of intermediate layers (SZ), adhesive layers (SK) and mixtures thereof.

For example, the films (F) can have further intermediate layers (SZ) in order to increase the mechanical stability. Intermediate layers can also prevent or minimize the permeation of polymers or residual monomers from a polymer layer into the cosmetic composition (KM).

In addition, to increase the bond strength, the films can also comprise one or more adhesive layers (SK) to reduce or prevent delamination (that is, flaking or formation of air space) between two layers.

A particularly preferred product as contemplated herein is exemplified in that the multi-layer film (F) additionally contains, in addition to the first polymer layer (P1), the second polymer layer (P2) and the barrier layer (BS), yet one or more further layers which are selected from intermediate layers (SZ) and/or adhesive layers (SK).

If the multi-layer film (F) also contains yet further layers in addition to the layers (P1), (P2) and (BS), suitable arrangements as contemplated herein of the layers are described below (considered from interior (in contact with the cosmetic composition (KM)) to the outside):
a) *Interior*-layer (P1)-first adhesive layer (SK1)-layer (P2)-second adhesive layer (SK2)-barrier layer (BS)-*outside*,
b) *Interior*-layer (P1)-adhesive layer (SK1)-layer (P2)-barrier layer (BS)-*outside*,
c) *Interior*-layer (P1)-layer (P2)-second adhesive layer (SK2)-barrier layer (BS)-*outside*,
d) *Interior*-barrier layer (BS)-first adhesive layer (SK1)-layer (P1)-second adhesive layer (SK2)-layer (P2)-*outside*,
e) *Interior*-barrier layer (BS)-adhesive layer (SK)-layer (P1)-layer (P2)-*outside*,
f) *Interior*-barrier layer (BS)-layer (Si)-adhesive layer (SK)-layer (P2)-*outside*,
g) *Interior*-layer (P1)-first adhesive layer (SK1)-barrier layer (BS)-second adhesive layer (SK2)-layer (P2)-*outside*,
h) *Interior*-layer (P1)-adhesive layer (SK)-barrier layer (BS)-layer (P2)-*outside*,
i) *Interior*-layer (P1)-barrier layer (BS)-adhesive layer (SK)-layer (P2)-*outside*

The product as contemplated herein comprises, as a second constituent, a cosmetic composition (KM) which is packaged in the package (VP) and comprises at least one oxidizing agent, a special thickening agent and an anionic surfactant.

The purposeful use of the product as contemplated herein is oxidative color change. For this purpose, as described above, a cosmetic composition (KM) containing an oxidizing agent is usually mixed with a second packaged preparation (B) assembled separately from (KM). The ready-to-use oxidative color-changing agent is prepared in this way. Depending on whether whitening, lightening or dyeing is to be achieved with the oxidative color change, the preparation (B) can contain various ingredients. If a pure lightening or whitening is to be achieved, the preparation contains (B) at least one alkalizing agent. If an oxidative dyeing is desired, oxidation dye precursors are often present in the preparation (B) in addition to the alkalizing agent. In order to ensure a sufficiently rapid miscibility of the preparations (KM) and (B), the preparation (KM) and in the preparation (B) are usually both flowable, aqueous or water-containing preparations.

The preparation (KM) as contemplated herein is an aqueous preparation. The water content of the preparation (KM) can, based on the total weight of the preparation (KM), be, for example, from about 60 to about 97% by weight, preferably from about 75 to about 93% by weight, preferably from about 78 to about 91% by weight, in particular at from about 80 to about 88.0% by weight. All weight specifications in % by weight refer to the total weight of water contained in the preparation (KM), which is related to the total weight of the preparation (KM).

The cosmetic composition (KM) contains at least one oxidizing agent as a first essential ingredient a). Preference is given to using certain oxidizing agents. It is therefore advantageous within the context of the present disclosure when the cosmetic composition (KM) contains at least one oxidizing compound selected from the group of persulfates, chlorites, hydrogen peroxide and addition products of hydrogen peroxide to urea, melamine and sodium borate, in particular hydrogen peroxide. The use of hydrogen peroxide has proved to be particularly advantageous as contemplated herein.

The concentration of the oxidizing agent in the composition (KM) is determined, on the one hand, by the legal requirements and, on the other hand, by the desired effect; preferably from about 0.5 to about 20.0% by weight solutions in water are used. It is therefore preferred as contemplated herein when the cosmetic composition (KM) contains the at least one oxidizing compound, in particular hydrogen peroxide, in a total amount of from about 0.5 to about 20% by weight, preferably from about 1.0 to about 18% by weight, more preferably from about 1.2 to about 16% by weight, in particular from about 1.5 to about 15% by weight, based on the total weight of the cosmetic composition (KM). The higher the content of oxidizing agent, in particular hydrogen peroxide, in the composition (KM), the greater the amount of gas produced when there is a proportionate decomposition of the oxidizing agent. Higher concentration oxidizing agent-containing preparations are therefore much more difficult to assemble in a storage-stable manner in a package (VP) than less concentrated preparations. The amount of hydrogen peroxide in this case refers to about 100% hydrogen peroxide.

In the course of work leading to this present disclosure, it has been found that the product as contemplated herein is particularly suitable for packaging and stable storage of highly concentrated hydrogen peroxide preparations (KM). Thus, packages (VP) as contemplated herein which contained preparations (KM) having from about 9 to about 12% by weight of hydrogen peroxide showed no volume changes even after several weeks of storage at elevated temperature (that is, no swelling) and no unplanned openings (that is, the packages did not burst).

The cosmetic composition (KM) contains at least one C₈-C₃₀ alcohol as a second essential ingredient b). In this context, mixtures of linear C₁₄-C₁₈ alcohols have particularly proven themselves. Such mixtures, in combination with the further features c) to e) of composition (KM) lead to an excellent stabilization of the at least one oxidizing agent, in particular the hydrogen peroxide. It is therefore advantageous within the context of the present disclosure when the cosmetic composition (KM) contains at least one C₁₀-C₃₀ alcohol selected from the group of linear C₁₀-C₂₈ alcohols, linear C₁₂-C₂₆ alcohols, linear C₁₄-C₂₀ alcohols, linear C₁₄-C₁₈ alcohols and mixtures of the alcohols mentioned above, in particular a mixture of linear C₁₄-C₁₈ alcohols. In the context of the present disclosure, the mixture of cetyl alcohol and stearyl alcohol known under the name cetearyl alcohol, in particular a mixture of about 50% by weight of cetyl alcohol and about 50% by weight of stearyl alcohol, based on the total weight of the mixture, has proven to be particularly advantageous.

The at least one C₈-C₃₀ alcohol is preferably used in certain quantity ranges. Preferred embodiments of the present disclosure are therefore exemplified in that the cosmetic composition (KM) contains the at least one C₈-C₃₀ alcohol, in particular a mixture of linear C₁₄-C₁₈ alcohols, in a total amount of from about 0.10 to about 7.0% by weight, preferably from about 0.50 to about 6.5% by weight, more preferably from about 1.0 to about 6.0% by weight, in particular from about 1.5 to about 5.0% by weight, based on the total weight of the cosmetic composition (KM). The use of the total amounts mentioned above of the at least one C₈-C₃₀ alcohol, in particular the mixture of linear C₁₄-C₁₈ alcohols, in combination with the further constituents of the cosmetic composition (KM) leads to a particularly good stabilization of the oxidizing agent contained in this composition contained, in particular the hydrogen peroxide.

The cosmetic composition (KM) contains at least one anionic surfactant as a third essential ingredient c). The use of these surfactants ensures sufficient miscibility of the cosmetic (KM) with the preparation (B), which contains the oxidation dye precursors, and also ensures a high storage stability, since a precipitation of components of the cosmetic composition (KM) is avoided. Preferred embodiments of the present disclosure are therefore exemplified in that the cosmetic composition (KM) contains at least one anionic surfactant selected from the group of (i) C₈-C₁₈ alkyl ether sulfates with about 1 to about 10 moles of ethylene oxide per mole of C₈-C₁₈ alkyl ether sulfate and salts thereof, in particular of sodium salts of C₁₂-C₁₄ alkyl ether sulfates with 2 moles of ethylene oxide per mole of C₁₂-C₁₄ alkyl ether sulfate, (ii) C₈-C₁₈ alkyl sulfates, (iii) salts of linear and branched C₈-C₃₀ carboxylic acids, (iv) acyl sarcosides having from about 8 to about 24 carbon atoms in the acyl group, (v) acyl taurides having from about 8 to about 24 carbon atoms in the acyl group, (vi) acyl isethionates having from about 8 to about 24 carbon atoms in the acyl group, (vii) sulfosuccinic acid mono- and dialkyl esters having from about 8 to about 24 carbon atoms in the alkyl group and sulfosuccinic monoalkyl polyoxyethyl esters having from about 8 to about 24 carbon atoms in the alkyl group and from about 1 to about 6, preferably from about 1 to about 4 oxyethyl groups, (viii) linear alpha-olefin sulfonates having from about 8 to about 24 carbon atoms, (ix) alpha-sulfonecarboxylic acid methyl esters of carboxylic acids having from about 8 to about 30 carbon atoms, (x) alkyl and/or alkenyl ether phosphates, (xi) sulfonates of unsaturated carboxylic acids having from about 8 to about 24 carbon atoms and from about 1 to about 6 double bonds, (xii) C₈-C₃₀ ether carboxylates with from about 1 to about 10 moles of ethylene oxide per mole of C₈-C₃₀ ether carboxylate and (xiii) mixtures thereof, in particular sodium salts of C₁₆-C₁₈ alky sulfates. An anionic surfactant suitable in the context of the present disclosure is the compound Sodium Cetearyl Sulfate known under the INCI name (CAS no. 59186-41-3).

In order to ensure a sufficient dispersion of all ingredients of the cosmetic product (KM), the at least one anionic surfactant is preferably used in certain total amounts. It is therefore preferred in the context of the present disclosure when the cosmetic composition (KM) contains the at least one anionic surfactant, in particular sodium salts of C₁₆-C₁₈ alkyl sulfates, in a total amount of from about 0.10 to about 3.0% by weight, preferably from about 0.12 to about 2.5% by weight, more preferably from about 0.15 to about 2.0% by weight, in particular from about 0.20 to about 1.5% by weight, based on the total weight of the cosmetic composition (KM).

The cosmetic composition (KM) contains at least one nonionic surfactant as a fourth essential component d). The combination of anionic and nonionic surfactant achieves an excellent dispersion of the components of the cosmetic composition (KM) and thus a high storage stability. In addition, the use of such surfactant combinations leads to a good distributability, in particular miscibility, of the cosmetic composition (KM) with the preparation (B) which contains the oxidation dye precursors. It is therefore preferred within the context of the present disclosure when the cosmetic composition (KM) contains at least one nonionic surfactant selected from the group of (i) ethoxylated and/or propoxylated alcohols and carboxylic acids having from about 8 to about 30 carbon atoms and from about 2 to about 30 ethylene oxide and/or propylene oxide units per mole of alcohol, (ii) addition products of from about 30 to about 50 moles of ethylene oxide to castor oil and hydrogenated castor oil, (iii) alkylpolyglucosides of the formula R¹O-[G]ₚ in which R¹ stands for an alkyl and/or alkenyl radical having from about 4 to about 22 carbon atoms, G stands for a sugar radical having 5 or 6 carbon atoms and p stands for numbers from about 1 to about 10, (iv) monoethanolamides of carboxylic acids having from about 8 to about 30 carbon atoms and (v) mixtures thereof, in particular addition products of 40 moles of ethylene oxide to hydrogenated castor oil. In the formula R¹O-[G]ₚ, the index number p indicates the degree of oligomerization (DP), that is, the distribution of mono- and oligoglucosides, and stands for a number between about 1 and about 10. While p always has to be an integer in a given compound, and here can primarily assume the values p=1 to 6, the value p for a given alkyloligoglucoside is an analytically determined arithmetic quantity, which usually represents a fractional number. As contemplated herein, preference is given to using alkyl and/or alkenyl oligoglucosides having a mean degree of oligomerization p of from about 1.1 to about 3.0. From an application point of view, those alkyl and/or alkenyl oligoglucosides whose degree of oligomerization is less than about 1.7 and in particular between about 1.2 and about 1.7 are preferred. The alkyl or alkenyl radical R¹ can be derived from primary alcohols having from about 4 to about 20, preferably from about 8 to about 16 carbon atoms. Very particularly preferred as contemplated herein are alkyl oligoglucosides based on hardened C_{12/14} coconut alcohol having a DP of 1-3, as are commercially available, for example, under the INCI name “Coco-Glucoside”. In the context of the present disclosure, particularly preferably used nonionic surfactants are addition products of 40 moles of ethylene oxide with hydrogenated castor oil, in particular the compound known under the MCI name PEG-40 Hydrogenated Castor Oil (CAS no. 61788-85-0).

In order to ensure a sufficient dispersion of all ingredients of the cosmetic product (KM), the at least one nonionic surfactant is preferably used in certain total amounts. Preferred embodiments of the present disclosure are therefore exemplified in that the cosmetic composition (KM) contains the at least one nonionic surfactant, in particular addition products of 40 moles of ethylene oxide to hydrogenated castor oil, in a total amount of from about 0.10 to about 2.5% by weight, preferably from about 0.12 to about 2.0% by weight, more preferably from about 0.15 to about 1.8% by weight, in particular from about 0.30 to about 1.5% by weight, based on the total weight of the cosmetic composition (KM).

Furthermore, the cosmetic composition (KM) contains at least one liquid cosmetic oil as a fifth essential component e). The use of this cosmetic oil leads to an increased stabilization of the oxidizing agent, in particular of the hydrogen peroxide, since it is surrounded with the cosmetic oil on dispersion or emulsification and thus is protected from decomposition as a result of the reaction with further constituents of the cosmetic composition (KM). Certain cosmetic oils are preferably used in the context of the present disclosure. It is therefore advantageous as contemplated herein when the cosmetic composition (KM) contains at least one liquid cosmetic oil selected from the group of (i) esters of linear or branched saturated or unsaturated C₂-C₃₀ fatty alcohols with linear or branched saturated or unsaturated C₂-C₃₀ fatty acids, which can be hydroxylated, (ii) C₈-C₂₂ fatty alcohol esters of monohydric or polyhydric C₂-C₇ hydroxycarboxylic acids, the triethyl citrates, (iii) mono-, di- and triglycerides of linear or branched, saturated or unsaturated, optionally hydroxylated C₈-C₃₀ fatty acids, (iv) dicarboxylic acid esters of linear or branched C₂-C₁₀ alkanols, (v) symmetrical, asymmetrical or cyclic esters of carbonic acid with fatty alcohols, the esters of dimers of unsaturated C₁₂-C₂₂ fatty acids with monovalent, linear, branched and cyclic C₂-C₁₈ alkanols or C₂-C₆ alkanols, (vi) benzoates acid esters of linear or branched C₈-C₂₂ alkanols, such as benzoic acid C₁₂-C₁₅ alkyl esters, benzoic acid isostearyl ester and benzoic acid octyldodecyl ester, (vii) synthetic hydrocarbons, such as polyisobutene and polydecenes, (viii) hydrocarbons, such as paraffins, and (ix) mixtures thereof, in particular paraffin oil. In the context of the present disclosure, it has turned out to be particularly advantageous when paraffin oil is used as a liquid cosmetic oil, in particular the compound known under the INCI name Paraffinum Liquidum (CAS no. 8042-47-5). Preferred paraffin oils as contemplated herein have dynamic viscosities from about 20 to about 150 mPa*s at 20° C. (measured according to DIN 51562-1 from 1999).

The at least one liquid cosmetic oil, in particular the at least one paraffin oil, is preferably present in the cosmetic compositions (KM) in specific total amounts. Sufficient stabilization of the oxidizing agent, in particular the hydrogen peroxide, is achieved as a result. Preferred embodiments of the present disclosure are therefore exemplified in that the cosmetic composition (KM) contains the at least one liquid cosmetic oil, in particular paraffin oil, in a total amount of from about 0.10 to about 25% by weight, preferably from about 2.0 to about 24% by weight, more preferably from about 4.0 to about 22% by weight, in particular from about 5.0 to about 20% by weight, based on the total weight of the cosmetic composition (KM).

In the course of work leading to this disclosure, it has been found that the use of the aforementioned essential ingredients b) to e) ensures that the cosmetic composition (KM), which contains at least one oxidizing agent, can be assembled and stored in the special package (VP), without this package swelling or bursting, which package has a barrier layer with a passage barrier effect for gases and water vapor.

In this context, a very specific combination of the essential ingredients a) to e) of the cosmetic composition (KM) has been found to be advantageous. In a preferred embodiment, the product as contemplated herein is therefore exemplified in that the cosmetic composition (KM) contains hydrogen peroxide, a mixture of linear C₁₄-C₁₈ alcohols, a sodium salt of a C₁₆-C₁₈ alky sulfate, an addition product of 40 moles of ethylene oxide to hydrogenated castor oil and paraffin oil.

To further optimize storage stability, the aforementioned compounds are preferably used in certain quantitative ranges in the preparation (KM). Particularly preferred embodiments are therefore exemplified in that the cosmetic composition (KM) contains
a) from about 1.5 to about 15% by weight of hydrogen peroxide,
b) from about 1.5 to about 5.0% by weight of a mixture of linear C₁₄-C₁₈ alcohols,
c) from about 0.20 to about 1.5% by weight of a sodium salt of C₁₆-C₁₈ alkyl sulfates,
d) from about 0.30 to about 1.5% by weight of an addition product of 40 moles of ethylene oxide to hydrogenated castor oil and
e) from about 5.0 to about 20% by weight of paraffin oil, each based on the total weight of the cosmetic product (KM).

The cosmetic composition (KM) preferably has an acidic pH value in order to avoid or reduce decomposition of the oxidizing agent used, in particular of the hydrogen peroxide. It is therefore preferred in the context of the present disclosure when the cosmetic composition (KM) has a pH value (measured at 20° C.) of from about pH 1.5 to about pH 5.0, preferably of from about pH 2.0 to about pH 4.6, more preferably of from about pH 2.3 to about pH 4.5, in particular of from about pH 2.5 to about pH 4.0.

The preparation (KM) located in the package (VP) contains the essential ingredients in an aqueous or aqueous-alcoholic carrier, which can be, for example, a cream, an emulsion, a gel or also a surfactant-containing foaming solution. To adjust the desired properties of these dosage forms, the preparation (KM) can further contain additional active ingredients, auxiliaries and additives.

The preparation (KM) may, for example, additionally contain one or more acids for stabilizing the oxidizing agent used, in particular the hydrogen peroxide. It is therefore preferred within the context of the present disclosure when the cosmetic composition (KM) additionally contains at least one acid selected from the group of dipicolinic acid, citric acid, acetic acid, malic acid, lactic acid, tartaric acid, hydrochloric acid, phosphoric acid, pyrophosphoric acid and their salts, benzoic acid and its salts, 1-hydroxyethane-1,1-diphosphonic acid, ethylenediaminetetraacetic acid and its salts, sulfuric acid and mixtures, in particular a mixture of dipicolinic acid, disodium pyrophosphate, benzoic acid and their salts and 1-hydroxyethane-1,1-diphosphonic acid.

A particularly high stabilization of the oxidizing agent, in particular the hydrogen peroxide, is achieved when the aforementioned acids are used in certain quantitative ranges. It is therefore advantageous in this context when the at least one acid, in particular the mixture of dipicolinic acid, disodium pyrophosphate, benzoic acid and their salts and 1-hydroxyethane-1,1-diphosphonic acid are present in a total amount of from about 0.1 to about 3.0% by weight, preferably from about 0.5 to about 2.5% by weight, more preferably from about 0.8 to about 2.0% by weight, in particular from about 0.9 to about 1.5% by weight, based on the total weight of cosmetic composition (KM).

In the following tables, particularly preferred embodiments AF 1 to AF 32 of the cosmetic composition (KM) contained in the package (VP) are listed (all data specified in % by weight, unless stated otherwise).

| | AF 1 | AF 2 | AF 3 | AF 4 |
|---|---|---|---|---|
| Oxidizing agent | 0.5-20 | 1.0-18 | 1.2-16 | 1.5-15 |
| C₈-C₃₀ alcohol | 0.10-7.0 | 0.50-6.5 | 1.0-6.0 | 1.5-5.0 |
| Anionic surfactant | 0.10-3.0 | 0.12-2.5 | 0.15-2.0 | 0.20-1.5 |
| Nonionic surfactant | 0.10-2.5 | 0.12-2.0 | 0.15-1.8 | 0.30-1.5 |
| Liquid cosmetic oil | 0.10-25 | 2.0-24 | 4.0-22 | 5.0-20 |
| Cosmetic carrier¹⁾ | ad 100 | ad 100 | ad 100 | ad 100 |

| | AF 5 | AF 6 | AF 7 | AF 8 |
|---|---|---|---|---|
| Oxidizing agent²⁾ | 0.5-20 | 1.0-18 | 1.2-16 | 1.5-15 |
| C₈-C₃₀ alcohol | 0.10-7.0 | 0.50-6.5 | 1.0-6.0 | 1.5-5.0 |
| Anionic surfactant | 0.10-3.0 | 0.12-2.5 | 0.15-2.0 | 0.20-1.5 |
| Nonionic surfactant | 0.10-2.5 | 0.12-2.0 | 0.15-1.8 | 0.30-1.5 |
| Liquid cosmetic oil | 0.10-25 | 2.0-24 | 4.0-22 | 5.0-20 |
| Cosmetic carrier¹⁾ | ad 100 | ad 100 | ad 100 | ad 100 |

| | AF 9 | AF 10 | AF 11 | AF 12 |
|---|---|---|---|---|
| Oxidizing agent²⁾ | 0.5-20 | 1.0-18 | 1.2-16 | 1.5-15 |
| C₈-C₃₀ alcohol³⁾ | 0.10-7.0 | 0.50-6.5 | 1.0-6.0 | 1.5-5.0 |
| Anionic surfactant | 0.10-3.0 | 0.12-2.5 | 0.15-2.0 | 0.20-1.5 |
| Nonionic surfactant | 0.10-2.5 | 0.12-2.0 | 0.15-1.8 | 0.30-1.5 |
| Liquid cosmetic oil | 0.10-25 | 2.0-24 | 4.0-22 | 5.0-20 |
| Cosmetic carrier¹⁾ | ad 100 | ad 100 | ad 100 | ad 100 |

| | AF 13 | AF 14 | AF 15 | AF 16 |
|---|---|---|---|---|
| Oxidizing agent²⁾ | 0.5-20 | 1.0-18 | 1.2-16 | 1.5-15 |
| C₈-C₃₀ alcohol³⁾ | 0.10-7.0 | 0.50-6.5 | 1.0-6.0 | 1.5-5.0 |
| Anionic surfactant⁴⁾ | 0.10-3.0 | 0.12-2.5 | 0.15-2.0 | 0.20-1.5 |
| Nonionic surfactant | 0.10-2.5 | 0.12-2.0 | 0.15-1.8 | 0.30-1.5 |
| Liquid cosmetic oil | 0.10-25 | 2.0-24 | 4.0-22 | 5.0-20 |
| Cosmetic carrier¹⁾ | ad 100 | ad 100 | ad 100 | ad 100 |

| | AF 17 | AF 18 | AF 19 | AF 20 |
|---|---|---|---|---|
| Oxidizing agent²⁾ | 0.5-20 | 1.0-18 | 1.2-16 | 1.5-15 |
| C₈-C₃₀ alcohol³⁾ | 0.10-7.0 | 0.50-6.5 | 1.0-6.0 | 1.5-5.0 |
| Anionic surfactant⁴⁾ | 0.10-3.0 | 0.12-2.5 | 0.15-2.0 | 0.20-1.5 |
| Nonionic surfactant⁵⁾ | 0.10-2.5 | 0.12-2.0 | 0.15-1.8 | 0.30-1.5 |
| Liquid cosmetic oil | 0.10-25 | 2.0-24 | 4.0-22 | 5.0-20 |
| Cosmetic carrier¹⁾ | ad 100 | ad 100 | ad 100 | ad 100 |

| | AF 21 | AF 22 | AF 23 | AF 24 |
|---|---|---|---|---|
| Oxidizing agent²⁾ | 0.5-20 | 1.0-18 | 1.2-16 | 1.5-15 |
| C₈-C₃₀ alcohol³⁾ | 0.10-7.0 | 0.50-6.5 | 1.0-6.0 | 1.5-5.0 |
| Anionic surfactant⁴⁾ | 0.10-3.0 | 0.12-2.5 | 0.15-2.0 | 0.20-1.5 |
| Nonionic surfactant⁵⁾ | 0.10-2.5 | 0.12-2.0 | 0.15-1.8 | 0.30-1.5 |
| Liquid cosmetic oil⁶⁾ | 0.10-25 | 2.0-24 | 4.0-22 | 5.0-20 |
| Cosmetic carrier¹⁾ | ad 100 | ad 100 | ad 100 | ad 100 |

| | AF 25 | AF 26 | AF 27 | AF 28 |
|---|---|---|---|---|
| Oxidizing agent²⁾ | 0.5-20 | 1.0-18 | 1.2-16 | 1.5-15 |
| C₈-C₃₀ alcohol³⁾ | 0.10-7.0 | 0.50-6.5 | 1.0-6.0 | 1.5-5.0 |
| Anionic surfactant⁴⁾ | 0.10-3.0 | 0.12-2.5 | 0.15-2.0 | 0.20-1.5 |
| Nonionic surfactant⁵⁾ | 0.10-2.5 | 0.12-2.0 | 0.15-1.8 | 0.30-1.5 |
| Liquid cosmetic oil⁶⁾ | 0.10-25 | 2.0-24 | 4.0-22 | 5.0-20 |
| Acid | 0.1-3.0 | 0.5-2.5 | 0.8-2.0 | 0.9-1.5 |
| Cosmetic carrier¹⁾ | ad 100 | ad 100 | ad 100 | ad 100 |

| | AF 29 | AF 30 | AF 31 | AF 32 |
|---|---|---|---|---|
| Oxidizing agent²⁾ | 0.5-20 | 1.0-18 | 1.2-16 | 1.5-15 |
| C₈-C₃₀ alcohol³⁾ | 0.10-7.0 | 0.50-6.5 | 1.0-6.0 | 1.5-5.0 |
| Anionic surfactant⁴⁾ | 0.10-3.0 | 0.12-2.5 | 0.15-2.0 | 0.20-1.5 |
| Nonionic surfactant⁵⁾ | 0.10-2.5 | 0.12-2.0 | 0.15-1.8 | 0.30-1.5 |
| Liquid cosmetic oil⁶⁾ | 0.10-25 | 2.0-24 | 4.0-22 | 5.0-20 |
| Acid⁷⁾ | 0.1-3.0 | 0.5-2.5 | 0.8-2.0 | 0.9-1.5 |
| Cosmetic carrier¹⁾ | ad 100 | ad 100 | ad 100 | ad 100 |

¹) aqueous or aqueous-alcoholic carrier
²) hydrogen peroxide, calculated on 100% hydrogen peroxide,
³) mixture of linear C₁₄-C₁₈ alcohols, in particular cetearyl alcohol,
⁴) sodium salts of C₁₆-C₁₈ alkysulfates, in particular sodium cetearyl sulfate,
⁵) addition products of 40 moles of ethylene oxide to hydrogenated castor oil,
⁶) paraffin oil,
⁷) mixture of dipicolinic acid, disodium pyrophosphate, benzoic acid and their salts and 1-hydroxyethane-1,1-diphosphonic acid.

The aforementioned embodiments AF 1 to 32 are respectively packaged in packages (VP) which have the below-described arrangement of the multi-layered film (F) (considered from interior (in contact with the cosmetic composition (KM)) to the outside):
a) *Interior*-layer (P1)-layer (P2)-barrier layer (BS)-*outside*,
b) *Interior*-layer (P1)-barrier layer (BS)-layer (P2)-*outside*,
c) *Interior*-layer (P2)-layer (P1)-barrier layer (BS)-*outside*,
d) *Interior*-layer (P2)-barrier layer (BS)-layer (P1)-*outside*,
e) *Interior*-barrier layer (BS)-layer (P1)-layer (P2)-*outside*,
f) *Interior*-barrier layer (BS)-layer (P2)-layer (P1)-*outside*,
g) *Interior*-layer (P1)-first adhesive layer (SK1)-layer (P2)-second adhesive layer (SK2)-barrier layer (BS)-*outside*,
h) *Interior*-layer (P1)-adhesive layer (SK1)-layer (P2)-barrier layer (BS)-*outside*,
i) *Interior*-layer (P1)-layer (P2)-second adhesive layer (SK2)-barrier layer (BS)-*outside*,
j) *Interior*-barrier layer (BS)-first adhesive layer (SK1)-layer (P1)-second adhesive layer (SK2)-layer (P2)-*outside*,
k) *Interior*-barrier layer (BS)-adhesive layer (SK)-layer (P1)-layer (P2)-*outside*,
l) *Interior*-barrier layer (BS)-layer (S1)-adhesive layer (SK)-layer (P2)-*outside*,
m) *Interior*-layer (P1)-first adhesive layer (SK1)-barrier layer (BS)-second adhesive layer (SK2)-layer (P2)-*outside*,
n) *Interior*-layer (P1)-adhesive layer (SK)-barrier layer (BS)-layer (P2)-*outside*,
o) *Interior*-layer (P1)-barrier layer (BS)-adhesive layer (SK)-layer (P2)-*outside*.

The products as contemplated herein obtainable in this way have a high storage stability and a water loss within the acceptable range during storage. No swelling or delamination of the package (VP) during storage of these cosmetic products as contemplated herein was observed.

The product as contemplated herein is used for the purpose of oxidative color change. For this purpose, the preparation (KM) packed in the package (VP), which is the oxidizing agent preparation, is mixed with at least one further preparation (B) to produce the ready-to-use color-changing agent. To prevent incompatibilities or to avoid a premature reaction, the preparations (KM) and (B) are assembled separately from each other.

A particularly preferred product as contemplated herein comprises a preparation (B) assembled separately from the preparation (KM), wherein the preparation (B) contains at least one compound selected from oxidation dye precursors, direct acting dyes, alkalizing agents and mixtures thereof. Preferred products of the present disclosure are therefore exemplified by additionally comprising at least one second cosmetic composition (KM2) which contains at least one compound selected from oxidation dye precursors, direct acting dyes, alkalizing agents and mixtures thereof and which is assembled separately from the cosmetic composition (KM).

If an oxidative dyeing is desired, preparation (B) contains at least one oxidation dye precursor. Oxidation dye precursors can be subdivided into developers and couplers, wherein the developers are used mostly in the form of their physiologically compatible salts (for example, in the form of their hydrochlorides, hydrobromides, hydrogen sulfates or sulfates) based on their greater sensitivity with respect to oxygen. Coupler components do not alone form significant dyeing in the context of oxidative dyeing, but always require the presence of developer components. Preferably, such agents contain at least one developer-type oxidation dye precursor and at least one coupler-type oxidation dye precursor. Particularly suitable developer-type oxidation dye precursors are selected in this case from at least one compound from the group formed from p-phenylenediamine, p-toluenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl] amine, N,N′-bis-(2-hydroxyethyl)-N,N′-bis-(4-aminophenyl)-1,3-diamino-propan-2-ol, bis-(2-hydroxy-5-aminophenyl) methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl) phenol, 4-amino-2-(diethylaminomethyl) phenol, 4,5-diamino-1-(2-hydroxyethyl) pyrazole, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo[1,2-a]pyrazol-1-one and their physiologically compatible salts.

Particularly suitable coupler-type oxidation dye precursors are selected from the group formed from 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy) ethanol, 1,3-bis(2,4-diaminophenoxy) propane, 1-methoxy-2-amino-4-(2-hydroxyethylamino) benzene, 1,3-bis(2,4-diaminophenyl) propane, 2,6-bis(2′-hydroxyethylamino)-1-methylbenzene, 2-({3-[(2-hydroxyethyl)amino]-4-methoxy-5-methylphenyl}amino) ethanol, 2({3-[(2-hydroxyethyl)amino]-2-methoxy-5-methylphenyl}amino) ethanol, 2-({3-[(2-hydroxyethyl)amino]-4,5-dimethylphenyl}amino) ethanol, 2-[3-morpholine-4-ylphenyl)amino] ethanol, 3-amino-4-(2-methoxyethoxy)-5-methylphenylamine, 1-amino-3-bis-(2-hydroxyethyl) aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline or mixtures of these compounds or their physiological compatible salts.

In addition, the preparation (B) can also contain yet one or more direct acting dyes. Suitable nonionic direct acting dyes can be selected from the group HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, 1,4-diamino-2-nitrobenzene, 2-amino-4-nitrophenol, 1,4-bis-(2-hydroxyethyl)-amino-2-nitrobenzene, 3-nitro-4-(2-hydroxyethyl) aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino] benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

Suitable anionic direct acting dyes can be selected from the group of Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, bromophenol blue and tetrabromophenol blue.

Suitable cationic direct acting dyes are cationic triphenylmethane dyes such as Basic Blue 7, Basic Blue 26, Basic Violet 2 and Basic Violet 14, aromatic systems which are substituted with a quaternary nitrogen group such as Basic Yellow 57, Basic Red 76, Basic Blue 99, Basic Brown 16 and Basic Brown 17, cationic anthraquinone dyes such as HC Blue 16 (Bluequat B) and direct acting dyes which contain a heterocycle having at least one quaternary nitrogen atom, in particular Basic Yellow 87, Basic Orange 31 and Basic Red 51. The cationic direct acting dyes that are sold under the trademark Arianor are also suitable cationic direct acting dyes as contemplated herein.

Dyeing processes on keratin fibers usually take place in an alkaline medium. However, in order to preserve the keratin fibers and the skin as much as possible, the setting of too high a pH value is not desirable. Therefore, it is preferred when the pH value of the agent (B) is between 7 and 11, in particular between 8 and 10.5. For the purposes of the present disclosure, the pH values are pH values that were measured at a temperature of about 22° C.

The preparation (B) can contain at least one alkalizing agent. The alkalizing agents which can be used as contemplated herein for adjusting the preferred pH value can be selected from the group of ammonia, alkanolamines, basic amino acids and inorganic alkalizing agents such as (earth) alkali metal hydroxides, (earth) alkali metal metasilicates, (earth) alkaline metal phosphates and (earth) alkali metal hydrogen phosphates. Preferred inorganic alkalizing agents are magnesium carbonate, sodium hydroxide, potassium hydroxide, sodium silicate and sodium metasilicate. Organic alkalizing agents which can be used as contemplated herein are preferably selected from monoethanolamine, 2-amino-2-methylpropanol and triethanolamine. The basic amino acids which can be used as alkalizing agents as contemplated herein are preferably selected from the group formed from arginine, lysine, ornithine and histidine, more preferably arginine. However, it has been found in the context of the investigations on the present disclosure that further preferred agents as contemplated herein are exemplified in that they additionally contain an organic alkalizing agent. An embodiment of the first subject of the present disclosure is exemplified in that the agent additionally contains at least one alkalizing agent which is selected from the group which is formed from ammonia, alkanolamines and basic amino acids, in particular ammonia, monoethanolamine and arginine or its compatible salts.

The preparation (B) can further contain additional active ingredients, auxiliaries and additives. For example, one or more fat components from the group of C₁₂-C₃₀ fatty alcohols, C₁₂-C₃₀ fatty acid triglycerides, C₁₂-C₃₀ fatty acid monoglycerides, the C₁₂-C₃₀ fatty acid diglycerides and/or the hydrocarbons can be included.

Preferably, in the preparation (B), a surface-active substance can additionally be added, wherein such surface-active substances are referred to as surfactants or as emulsifiers, depending on the field of application: They are preferably selected from anionic, zwitterionic, amphoteric and nonionic surfactants and emulsifiers.

Preferably, the preparation (B) contains at least one anionic surfactant. Preferred anionic surfactants are fatty acids, alkyl sulfates, alkyl ether sulfates and ether carboxylic acids having from about 10 to about 20 carbon atoms in the alkyl group and up to about 16 glycol ether groups in the molecule.

Furthermore, the preparation (B) can additionally contain at least one zwitterionic surfactant. Preferred zwitterionic surfactants are betaines, N-alkyl-N,N-dimethylammonium glycinates, N-acyl-aminopropyl-N,N-dimethylammonium glycinates, and 2-alkyl-3-carboxymethyl-3-hydroxyethyl-imidazolines. A preferred zwitterionic surfactant is known by the INCI name Cocamidopropyl Betaine.

In addition, it can be provided that the preparation (B) contains at least one amphoteric surfactant. Preferred amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids. Particularly preferred amphoteric surfactants are N-cocoalkylaminopropionate, cocoacylaminoethylaminopropionate and C₁₂-C₁₈ acylsarcosine.

Furthermore, it has proved to be advantageous when the preparation (B) contains further nonionic surface active substances. Preferred nonionic surfactants are proven alkyl polyglycosides and alkylene oxide addition products to fatty alcohols and fatty acids with in each case from about 2 to about 30 moles of ethylene oxide per mole of fatty alcohol or fatty acid. Preparations having excellent properties are also obtained when they contain fatty acid esters of ethoxylated glycerol as nonionic surfactants.

The nonionic, zwitterionic or amphoteric surfactants are used in proportions of from about 0.1 to about 45% by weight, preferably from about 1 to about 30% by weight and very particularly preferably from about 1 to about 15% by weight, based on the total weight of the preparation (B).

The preparation (B) can additionally contain at least one thickening agent. There are no fundamental restrictions with regard to these thickening agents. Both organic and purely inorganic thickening agents can be used. Suitable thickening agents are anionic, synthetic polymers, cationic synthetic polymers, naturally occurring thickening agents such as nonionic guar gums, scleroglucan gums or xanthan gums, gum arabic, ghatti gum, karaya gum, tragacanth gum, carrageenan gum, agar-agar, locust bean gum, pectins, alginates, starch fractions and derivatives such as amylose, amylopectin and dextrins, and cellulose derivatives such as methylcellulose, carboxyalkylcelluloses and hydroxyalkylcelluloses, nonionic fully synthetic polymers such as polyvinyl alcohol or polyvinylpyrrolidinone; and inorganic thickening agents, in particular phyllosilicates such as bentonite, in particular smectites, such as montmorillonite or hectorite.

Further, the preparation (B) can contain other active ingredients, auxiliaries and additives, such as nonionic polymers such as vinylpyrrolidinone/vinyl acrylate copolymers, polyvinylpyrrolidinone, vinylpyrrolidinone/vinyl acetate copolymers, polyethylene glycols and polysiloxanes; additional silicones, such as volatile or nonvolatile, straight-chain, branched or cyclic, crosslinked or uncrosslinked polyalkylsiloxanes (such as dimethicones or cyclomethicones), polyarylsiloxanes and/or polyalkylarylsiloxanes, in particular polysiloxanes with organofunctional groups, such as substituted or unsubstituted amines (amodimethicones), carboxyl, alkoxy and/or hydroxyl groups (dimethicone copolyols), linear polysiloxane (A) polyoxyalkylene (B) block copolymers, grafted silicone polymers; cationic polymers such as quaternized cellulose ethers, polysiloxanes with quaternary groups, dimethyldiallylammonium chloride polymers, acrylamide-dimethyldiallyl-ammonium chloride copolymers, with diethyl sulfate quaternized dimethylaminoethylmethacrylate-vinylpyrrolidinone copolymers, vinylpyrrolidinone-imidazolinium methochloride copolymers, and quaternized polyvinyl alcohol; zwitterionic and amphoteric polymers; anionic polymers such as polyacrylic acids or crosslinked polyacrylic acids; structurants such as glucose, maleic acid and lactic acid, hair conditioning compounds such as phospholipids, for example, lecithin and cephalins; perfume oils, dimethylisosorbide and cyclodextrins; active ingredients that improve fiber structure, in particular mono-, di- and oligosaccharides such as, glucose, galactose, fructose, fruit sugar and lactose; dyes for staining the agent; anti-dandruff active substances such as piroctone olamine, zinc omadine and climbazole; amino acids and oligopeptides; protein hydrolyzates based on animal and/or vegetable, and in the form of their fatty acid condensation products or optionally anionically or cationically modified derivatives; fatty substances and vegetable oils; sunscreens and UV blockers; active ingredients such as panthenol, pantothenic acid, pantolactone, allantoin, pyrrolidinonecarboxylic acids and their salts, and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; fats and waxes such as fatty alcohols, beeswax, montan wax and paraffins; swelling and penetrating substances such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescing agents such as ethylene glycol mono- and distearate and PEG-3-distearate and pigments.

The selection of these further substances is made by the person skilled in the art according to the desired properties of the preparation (B) and of the product as contemplated herein. With regard to further optional components and the amounts of these components used, reference is expressly made to the relevant manuals known to the person skilled in the art. The additional active ingredients and auxiliaries are preferably used in the preparation (B) in amounts of from about 0.0001 to about 25% by weight, in particular from about 0.0005 to about 15% by weight, in each case based on the total weight of the preparation (B).

The following examples illustrate the present disclosure without, however, limiting it:

### Examples

A 100 nm thick layer of silicon dioxide SiOx was vapor-deposited on a film layer of polyethylene terephthalate with a thickness of 12 μm (microns). Subsequently, the SiOx layer was overcoated with about 3 g/m2 ORMOCER polymer and cured. A 70 μm (microns) thick layer of polypropylene was then applied to the ORMOCER layer. A package (VP) was produced from the film.

The following cosmetic composition (KM) was used (all specification in % by weight).

| Ingredients | KM |
|---|---|
| Potassium hydroxide (50%) | 0.24 |
| Sodium benzoate | 0.040 |
| Dipicolinic acid | 0.10 |
| Disodiumpyrophosphate | 0.10 |
| 1-hydroxyethane-1,1-diphosphonic acid (60%) | 0.31 |
| Oxidizing agent¹⁾ | 12 |
| C₈-C₃₀ alcohol²⁾ | 3.37 |
| Anionic surfactant³⁾ | 0.45 |
| Nonionic surfactant⁴⁾ | 0.68 |
| Liquid cosmetic oil⁵⁾ | 17 |
| Water | ad 100 |

¹⁾preferably hydrogen peroxide, calculated on 100% H₂O₂,
²⁾preferably a mixture of linear C₁₄-C₁₈ alcohols, in particular cetearyl alcohol,
³⁾preferably a sodium salt of C₁₆-C₁₈ alkysulfates, in particular sodium cetearyl sulfate,
⁴⁾preferably an addition product of 40 moles of ethylene oxide to hydrogenated castor oil, in particular PEG-40 hydrogenated castor oil,
⁵⁾preferably paraffin oil

The cosmetic composition KM filled into the previously described package (VP). Then the packages were stored at 40° C. for 24 weeks. The packages were not swollen or delaminated.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product for changing the natural color of keratinic fibers comprising
(i) a package, comprising a multi-layer film, which comprises a first polymer layer, a second polymer layer and a barrier layer, and
(ii) a cosmetic composition packed in the package and comprising:
a) an oxidizing compound,
b) a C₈-C₃₀ alcohol,
c) an anionic surfactant,
d) a nonionic surfactant and
e) a liquid cosmetic oil.

2. The cosmetic product according to claim 1, wherein the multi-layer film comprises the barrier layer disposed between the first polymer layer and the second polymer layer.

3. The cosmetic product according to claim 1, wherein the first polymer layer comprises polypropylene, polyethylene, polyester, polyamide or polyvinyl alcohol.

4. The cosmetic product according to claim 1, wherein the first polymer layer has a layer thickness of from about 20.0 to about 300 μm.

5. The cosmetic product according to claim 1, wherein the second polymer layer comprises polyethylene terephthalate or polyethylene naphthalate, in particular from polyethylene terephthalate.

6. The cosmetic product according to claim 1, wherein the second polymer layer (P2) has a layer thickness of from about 1.00 to about 100 μm.

7. The cosmetic product according to claim 1, wherein the barrier layer comprises aluminum oxides, magnesium oxides, silicon oxides, titanium oxides, tin oxides, zirconium oxides, inorganic-organic hybrid polymers (ORMOCER polymers) or mixtures thereof, in particular from silicon oxides.

8. The cosmetic product according to claim 1, wherein the barrier layer has a layer thickness of from about 1.00 nm to about 1000 nm.

9. The cosmetic product according to claim 1, wherein the cosmetic composition comprises the oxidizing compound, in a total amount of from about 0.5 to about 20% by weight.

10. The cosmetic product according to claim 1, wherein the cosmetic composition comprises the C₈-C₃₀ alcohol, in a total amount of from about 0.10 to about 7.0% by weight.

11. The cosmetic product as claimed in claim 1, wherein the C₈-C₃₀ alcohol comprises a mixture of linear C₁₄-C₁₈ alcohols and is present in an amount of from about 1.5 to about 5.0% by weight, with respect to the total weight of the cosmetic composition.

12. The cosmetic product as claimed in claim 1, wherein the oxidizing compound comprises hydrogen peroxide and is present in a total quantity of from about 1.5 to about 15% by weight, in relation to the total weight of the cosmetic composition.

13. The cosmetic product as claimed in claim 1, wherein:
the C₈-C₃₀ alcohol comprises a mixture of linear C₁₄-C₁₈ alcohols and is present in an amount of from about 1.5 to about 5.0% by weight, with respect to the total weight of the cosmetic composition;
the oxidizing compound comprises hydrogen peroxide and is present in a total quantity of from about 1.5 to about 15% by weight, in relation to the total weight of the cosmetic composition;
wherein the cosmetic composition further comprises water—with respect to the total weight of the cosmetic composition—in a quantity of from about 60 to about 97% by weight.

14. The cosmetic product as claimed in claim 13, wherein the cosmetic composition further comprises:
an inorganic alkalizing agent; and
a mixture of acids comprising:
at least one of dipicolinic acid, disodium pyrophosphate, ethylenediamine tetraacetic acid, or salts thereof;
1-hydroxyethane-1,1-diphosphonic acid; and
benzoic acid or a salt thereof;
wherein the acids are present in a total quantity of from about 0.9 to about 1.5% by weight, in relation to the total weight of the cosmetic composition.

15. The cosmetic product as claimed in claim 14, wherein the cosmetic composition further comprises:
sodium cetearyl sulfate present in a total quantity of from about 0.20 to about 1.5% by weight, based on the total weight of the cosmetic composition; and
PEG-40 hydrogenated castor oil present in a total quantity of from about 0.30 to about 1.5% by weight, based on the total weight of the cosmetic composition.

16. The cosmetic product as claimed in claim 1, wherein:
the first polymer layer comprises polypropylene and has a layer thickness of from about 60.0 μm to about 90.0 μm; and
the second polymer layer comprises polyethylene terephthalate and has a layer thickness of from about 10.0 μm to about 20.0 μm.

17. The cosmetic product as claimed in claim 1, wherein the barrier layer is a multi-layer film comprising a layer comprising silicon dioxide and a layer comprising an inorganic-organic hybrid polymer.

18. The cosmetic product as claimed in claim 1, The cosmetic product as claimed in claim 1, wherein:
the first polymer layer comprises polypropylene and has a layer thickness of from about 60.0 μm to about 90.0 μm;
the second polymer layer comprises polyethylene terephthalate and has a layer thickness of from about 10.0 μm to about 20.0 μm; and
the barrier layer comprises:
a layer comprising silicon dioxide; and
a layer comprising an inorganic-organic hybrid polymer;
wherein the barrier layer has a layer thickness of from about 10.0 nm to 150 nm and is disposed between the first polymer layer and the second polymer layer.

19. A cosmetic product for changing the natural colour of keratinous fibres comprising:
(i) a package, comprising a multi-layer film, which comprises:
a first polymer layer comprising polypropylene and having a layer thickness of from about 60.0 μm to about 90.0 μm,
a second polymer layer comprising polyethylene terephthalate and having a layer thickness of from about 10.0 μm to about 20.0 μm, and
a barrier layer comprising:
a layer comprising silicon dioxide; and
a layer comprising an inorganic-organic hybrid polymer;
wherein the barrier layer has a layer thickness of from about 10.0 nm to 150 nm and is disposed between the first polymer layer and the second polymer layer, and
(ii) a cosmetic composition, which is packaged in the package and comprises:
a) an oxidizing compound comprising hydrogen peroxide and present in a total quantity of from about 1.5 to about 15% by weight, in relation to the total weight of the cosmetic composition,
b) a mixture of linear C₁₄-C₁₈ alcohols and is present in an amount of from about 1.5 to about 5.0% by weight, with respect to the total weight of the cosmetic composition;
c) sodium cetearyl sulfate present in a total quantity of from about 0.20 to about 1.5% by weight, based on the total weight of the cosmetic composition;
d) PEG-40 hydrogenated castor oil present in a total quantity of from about 0.30 to about 1.5% by weight, based on the total weight of the cosmetic composition;
e) a liquid cosmetic oil present in a total quantity of from about 5.0 to about 20% by weight, based on the total weight of the cosmetic composition;
f) water present in a total quantity of from about 60 to about 97% by weight, in relation to the total weight of the cosmetic composition.

20. A cosmetic product for changing the natural colour of keratinous fibres comprising:
(i) a package, comprising a multi-layer film, which comprises:
a first polymer layer comprising polypropylene and having a layer thickness of from about 60.0 μm to about 90.0 μm,
a second polymer layer comprising polyethylene terephthalate and having a layer thickness of from about 10.0 μm to about 20.0 μm, and
a barrier layer comprising:
a layer comprising silicon dioxide; and
a layer comprising an inorganic-organic hybrid polymer;
wherein the barrier layer has a layer thickness of from about 10.0 nm to 150 nm and is disposed between the first polymer layer and the second polymer layer, and
(ii) a cosmetic composition, which is packaged in the package and consists of:
a) an oxidizing compound,
b) a mixture of linear C₁₄-C₁₈ alcohols;
c) an inorganic alkalizing agent;
d) sodium cetearyl sulfate;
e) PEG-40 hydrogenated castor oil;
f) a liquid cosmetic oil;
g) water present in a total quantity of from about 60 to about 97% by weight, in relation to the total weight of the cosmetic composition; and
h) a mixture of acids comprising:
at least one of dipicolinic acid, disodium pyrophosphate, ethylenediamine tetraacetic acid, or salts thereof;
1-hydroxyethane-1,1-diphosphonic acid; and
benzoic acid or a salt thereof.
